(19) United States
(12) Patent Application Publication
(10) Pub. No.: US 2011/0295211 A1
(43) Pub. Date: Dec. 1, 2011

(54) APPLICATOR

(76) Inventors: Don F. Yeager, Millville, NJ (US); Paul H. Andrews, Millville, NJ (US)

(21) Appl. No.: 12/791,328

(22) Filed: Jun. 1, 2010

(51) Int. Cl. A61M 35/00; A61M 5/31

(52) U.S. Cl. 604/187; 604/310

(57) ABSTRACT

An applicator for applying a substance to an intended surface. The applicator may include a main body including an elongated handle and an elongated tip configured to hold a predetermined amount of the substance. Further, the applicator may include an extension which extends radially from the main body in an area proximate to where the handle meets the tip and a breakaway section including an elongated sleeve which extends in a direction perpendicular to at least a portion of the tip and extends from the extension in an area proximate to a terminal end of the extension. Further, the elongated tip may extend from the handle in an area proximate to where the extension extends from the main body. Additionally, the applicator may be a single, integral piece such that the handle, the elongated tip, the extension and the breakaway section are integral with each other.

### FIELD OF THE DISCLOSURE

Aspects of the present disclosure generally relate to applicators. Particular aspects of the present disclosure relate to an applicator for applying a substance into a nasal cavity.

### BACKGROUND

Applicators are known in that art. Some conventional applicators include a mechanism that is designed to apply a particular amount of gel, cream, salve, etc.

### SUMMARY

The present disclosure generally relates to new and novel structures for applicators. For example, aspects of the disclosure relate to an applicator for applying a substance to an intended surface. The applicator may include a main body including an elongated handle and an elongated tip configured to hold a predetermined amount of the substance. Further, the applicator may include an extension which extends radially from the main body in an area proximate to where the handle meets the tip and a breakaway section including an elongated sleeve which extends in a direction perpendicular to at least a portion of the tip and extends from the extension in an area proximate to a terminal end of the extension. Further, the elongated tip may extend from the handle in an area proximate to where the extension extends from the main body. Additionally, the applicator may be a single, integral piece such that the handle, the elongated tip, the extension and the breakaway section are integral with each other.

Further aspects of the disclosure relate to an applicator for applying a substance to an intended surface. The applicator may include a main body including an elongated handle with a flat surface and an elongated tip with a flat surface configured to hold a predetermined amount of the substance. Further, the applicator may include an extension which extends radially from the main body in an area proximate to where the handle meets the tip and a breakaway section including an elongated sleeve which extends in a direction perpendicular to at least a portion of the tip and extends from the extension in an area proximate to a terminal end of the extension. Further, the elongated tip may extend from the handle in an area proximate to where the extension extends from the main body and the flat surface of the handle and the flat surface of the elongated tip may face the same direction and correspond to each other such that the flat surface on the handle indicates the direction that the flat surface of the elongated tip faces.

Additional aspects of the disclosure relate to an applicator for applying a substance to an intended surface. The applicator may include a main body including an elongated handle, an elongated tip configured to hold a predetermined amount of the substance and an elongated member. Further, the applicator may include an extension which extends radially from the main body in an area proximate to where the handle meets the tip and a breakaway section including an elongated sleeve which extends in a direction perpendicular to at least a portion of the tip and extends from the extension in an area proximate to a terminal end of the extension. Further, the elongated tip and the elongated member may both extend from the handle in an area proximate to where the extension extends from the main body to form a U or horseshoe shape with a gap extending between the elongated tip and the elongated member.

The above summary presents general aspects of the disclosure in order to provide a basic understanding of at least some of its aspects. The summary is not intended as an extensive overview of the disclosure. It is not intended to identify key or critical elements of the disclosure or to delineate the scope of the disclosure. The above summary merely presents some concepts of the disclosure in a general form as a prelude to the more detailed description provided below.

### BRIEF DESCRIPTION OF THE DRAWINGS

A more complete understanding of the present disclosure and certain advantages thereof may be acquired by referring to the following description in consideration with the accompanying drawings, in which like reference numbers indicate like features, and wherein:

FIG. 1A illustrates an applicator according to at least one aspect of the disclosure wherein portions of the applicator are shown in cross-section for illustrative purposes;

FIG. 1B illustrates the applicator shown in FIG. 1A according to at least one aspect of the disclosure;

FIGS. 2A, 2B 2C, 2D and 2E illustrate various embodiments of a tip of the applicator according to aspects of the disclosure;

FIGS. 3A-3D illustrate various views of another embodiment of an applicator according to at least one aspect of the disclosure, wherein portions of the applicator are shown in cross-section for illustrative purposes;

FIGS. 4A and 4B illustrate various views of another embodiment of an applicator according to at least one aspect of the disclosure, wherein portions of the applicator are shown in cross-section for illustrative purposes; and

FIG. 5 illustrates another embodiment of an applicator according to at least one aspect of the disclosure wherein portions of the applicator are shown in cross-section for illustrative purposes.

### DETAILED DESCRIPTION

In the following description of various example embodiments of the disclosure, reference is made to the accompanying drawings, which form a part hereof, and in which are shown by way of illustration various example structures and systems in which aspects of the disclosure may be practiced. It is to be understood that other specific arrangements of parts, structures, example devices, systems, and the like may be utilized and structural and functional modifications may be made without departing from the scope of the present disclosure. When the same reference number appears in more than one drawing, that reference number is used consistently in this specification and the drawings to refer to the same or similar parts throughout.

Initially, it is noted that, for the sake of brevity, throughout the disclosure the term “substance” may be used to refer to any substances that may be used in an applicator according to aspects of this disclosure. Such substances may include, but are not limited to: liquids, gels, lotions, creams, salves, etc.

Some circumstances may require applicators to reliably apply an accurate amount of a substance (i.e., a controlled dose). This may be especially true, when the controlled dose to be dispensed is a relatively small amount (e.g., in the range 0.05-0.6 milliliters). However, often a large amount of the desired controlled dose may remain on the applicator during application. Therefore, aspects of the disclosure are directed to applicators which accurately apply the desired amount of a substance, even if the controlled dose is relatively small (e.g., in the range 0.05-0.6 milliliters).

It is noted that the dispensing of such small amounts of substance is particularly common in the fields of science and medicine (e.g., to treat various maladies). The controlled dose may be extremely relevant to the treatment where too little substance may result in ineffective treatment or too much substance in serious side effects. Hence, it would be desirable to ensure that controlled dose is dispensed accurately and reliably. Therefore, an applicator according to aspects of disclosure may have particular usefulness in such fields. For example, aspects of the disclosure may relate to an applicator configured for applying an accurate and controlled dose of substance (e.g., a medicine or other chemical) in a nasal passageway.

Further aspects of the disclosure relate to applicators for applying a particular amount of gel, cream, salve, etc., wherein the structure of the applicator allows the gel, cream, salve, etc. contained in the applicator to be easily accessible, and, also, ensures that the gel, cream, salve, etc. is kept uncontaminated. Further, aspects of the disclosure relate to applicators which are easy to manipulate in order to apply the gel, cream, salve, etc. contained in the applicator to an intended surface, and, also, configured to reduce the likelihood of injury during use.

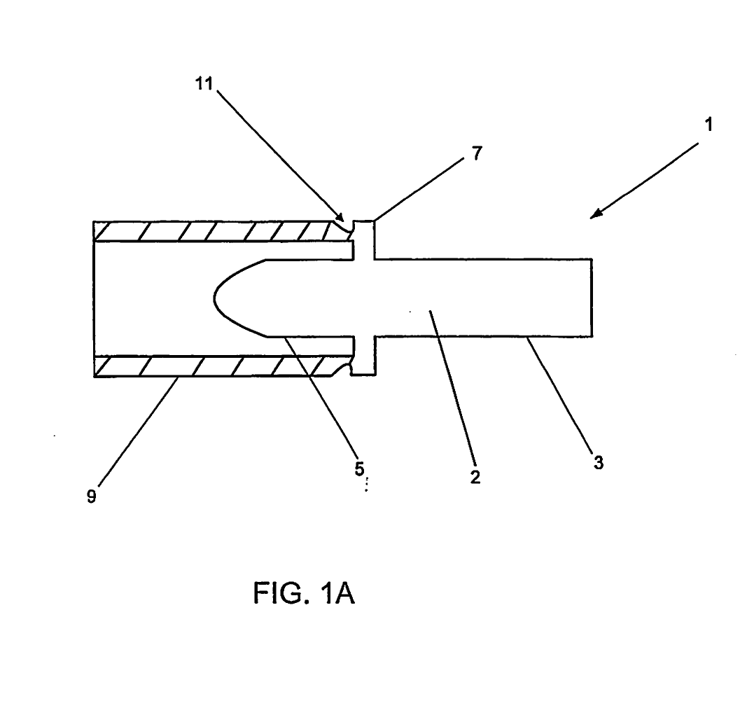

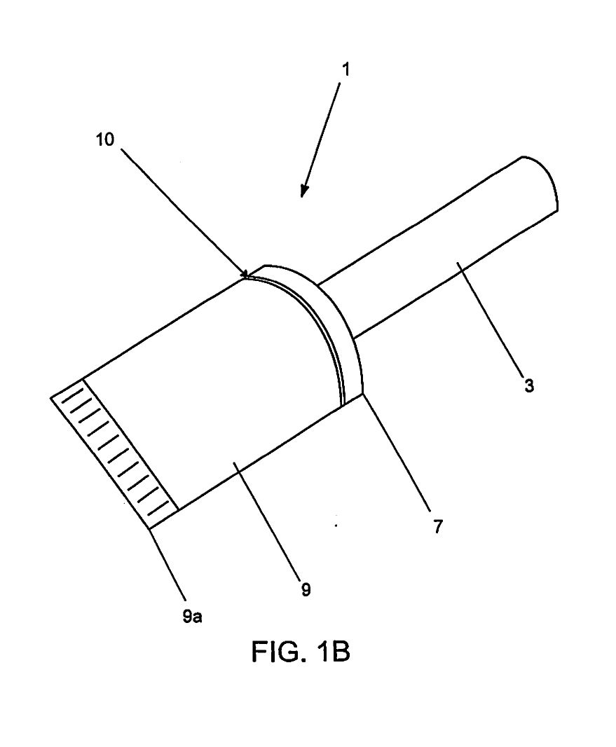

An illustrative embodiment of an apparatus for applying an accurate and controlled dose according to one aspect of the disclosure is shown at FIG. 1A. As shown in FIG. 1A, the applicator 1 may include a main body 2 with a handle 3 and a tip 5. Further, the applicator 1 may also include an extension 7. Additionally, the applicator 1 may include a breakaway section 9 and a separation section 11. Also, a substance (not shown) may be applied to the tip 5. In other words, the tip 5 may be coated in a substance that is to be applied by the applicator 1.

The operation of the applicator 1 is briefly described below. Initially, the user may grasp the handle 3 of the applicator 1 with a first hand and use their other hand to break off the breakaway section 9 (e.g., the user may twist the breakaway section 9 to sever it from the rest of the applicator 1) and dispose of it. When breakaway section 9 is broken off of the applicator 1, the tip 5 (which is coated with the substance to be applied) is exposed. Therefore, once a user removes the breakaway section 9, the user may maneuver the applicator 1 so that the tip 5 of the applicator 1 is in a position to apply the substance to the area that is intended to receive the substance. The user may then contact the intended area with the tip 5 of the applicator 1 to apply the substance to the intended area. According to aspects of the disclosure, the user may apply the substance to the intended area by maneuvering the tip 5 along the intended area (e.g., twisting, rotating, wiping, scraping, etc. the tip 5 along the intended area).

Given the general description of various example aspects of the disclosure provided above, more detailed descriptions of various specific example features of applicator structures according to the disclosure are provided below.

As discussed above, the applicator 1 may include a main body 2 with a handle 3 and a tip 5, an extension 7 and a removable breakaway section 9 attached to the applicator 1 (e.g., attached at the extension 7). According to aspects of the disclosure, the length of the applicator 1 may be in a range of 2.0-3.0 inches, including the breakaway section 9. Further, according to aspects of the disclosure, when the breakaway section 9 has been removed from the applicator 1, the length of the applicator 1 may be in a range of 1.5-2.5 inches.

According to some aspects of this disclosure, the applicator 1, or portions thereof, may be made of a plastic material such as polypropylene, high density polyethylene, low density polyethylene, polyethylene terephthalate (PET) or some other type of plastic. Of course, according to other aspects of the disclosure, the applicator 1, or portions thereof, may be made from other materials as well (e.g., metals, rubber, composites, alloys, etc.).

According to some aspects of the disclosure, the applicator 1 may be formed as a single, integral piece. For example, according to some embodiments of the disclosure, the entire applicator 1 including main body 2 which includes the handle 3 and the tip 5, the extension 7 and the breakaway section 9 may all be a single, integral component made from polypropylene. Such a one-piece, integral applicator 1 may be formed by conventional methods, such as injection molding and the like.

Many benefits may be achieved by configuring the applicator 1 as a single, integral piece. For example, if the applicator 1 is formed as a single, integral piece, then no assembly is required. In other words, if the applicator 1 does not have any separate components that must be engaged with each other to form the applicator 1, then the time, expense and effort associated with such an assembly may be eliminated. A further benefit of the single, integral applicator 1 is that it may reduce the potential for contamination. For example, if the applicator 1 is formed integrally, then the manufacturer would not have to assemble the applicator 1 and, thereby, potentially contaminate the parts of the applicator 1, such as the tip 5 during such an assembly process. Further, a single, integral one-piece applicator 1, may also simplify the process of filing the applicator 1 with the relevant substance.

According to some embodiments of this disclosure, the applicator 1 is made from a single material and the wall thicknesses of the different sections (e.g., the handle 3 and the breakaway section 9) are varied in order to provide appropriate rigidity. For example, according to one embodiment, in order to make the breakaway section 9 easier to detach from the applicator 1, the wall thickness of the portion of the breakaway section 9 that is near the extension 7 may be less thick than the wall thickness of other portions of the applicator 1, such as the handle 3, tip 5 and the remainder of the breakaway section 9. In contrast to the thinner portion of the breakaway section 9, the thicker wall sections of the handle 3, the tip 5 and the majority of the breakaway section 9 may be more rigid and, thereby, more durable. This may be advantageous because, for example, a more rigid handle 3 or tip 5 may prevent buckling. However, this is merely one embodiment. Of course, according to other embodiments, different portions of the applicator 1 may be made of different materials, have different thicknesses, different rigidities, etc. It is noted that aspects of the disclosure may be directed to a one-piece, disposable unit dose nasal applicator.

The above discussion is not meant to imply that the applicator 1 must be an integral one-piece applicator 1. On the contrary, according to aspects of the disclosure, the applicator 1 may be comprised of more than one type of material (e.g., as will be discussed in detail below, according to aspects of the disclosure, an applicator 1 may include different materials).

As seen in FIG. 1A, according to aspects of the disclosure, the handle 3 may be a post-style handle which is joined with, or integral with, the tip 5 and the extension 7. For example, as seen in FIG. 1A, according to aspects of the disclosure, the handle 3 may extend from the tip 5 in an area proximate to where the extension 7 extends from the main body 2. In the embodiment shown in FIG. 1A, the handle 3 is cylindrical, however, this is merely an illustrative embodiment and according to other embodiments of the disclosure (e.g., such as those embodiments described below), the handle 3 may be configured differently. According to aspects of the disclosure, the length of the handle 3 may be in a range of 1.5-2.0 inches. Further, according to aspects of the disclosure, the diameter, or thickness, of the handle 3 may be in a range of 0.2 inches and 0.5 inches. However, it is noted that, of course, the dimensions may be varied as desired.

As described above, according to aspects of the disclosure, the applicator 1 may include an extension 7. For example, as seen in FIG. 1A, according to aspects of the disclosure, the extension 7 may extend from the main body 2 of the applicator 1 at an area proximate to the area of the main body 2 wherein the handle 3 meets the tip 5. According to aspects of the disclosure, the extension 7 may extend substantially or completely around the circumference of the main body 2. For example, as seen in FIG. 1A, the extension is configured as a disk extending substantially around the circumference of the main body 2 at an area proximate to the area of the main body 2 wherein the handle 3 meets the tip 5. However, it is noted that the extension 7 may be configured differently in other embodiments. For example, the extension 7 may be angular. As another example, the extension 7 may be configured such that it resembles a cup that faces towards the breakaway section 9 (toward the left direction shown in FIG. 1A). Such configurations as described above may act as a stop to prevent a substance from migrating from the tip 5 to the handle 3. For example, depending on the viscosity of the substance applied to the tip 5, during the filling of the applicator 1 with the substance, during storage of the applicator 1, during use of the applicator 1, etc., the substance may tend to migrate from the tip 5 towards the handle 3. By providing the extension 7 to the main body 2, the extension 7 may function to contain the substance near the tip 5 and away from the handle 3.

According to aspects of the disclosure, the extension 7 may be configured to provide additional benefits. For example, according to aspects of the disclosure, the extension 7 may be configured to act as a safety feature during application of the substance to the intended surface. For example, according to aspects of the disclosure, the applicator 1 may be an applicator configured for applying a substance into the nasal cavity. In such an embodiment, the extension 7 may be sized to prevent the tip 5 from penetrating too far into the nasal cavity and causing injury. For example, the extension 7 may be sized to be too large to fit inside the nasal cavity and, thereby, the extension 7 provides a stop that prevents the tip 5 from over penetrating into the nasal cavity. This may be particularly advantageous if person was using the applicator 1 to apply a substance to another individual (e.g., in a doctor patient setting, parent child setting, etc.). According to aspects of the disclosure, the length of the extension 7 (e.g., in the direction extending radially away from the main body 2) may be in a range of 0.1-0.4 inches and the diameter of the extension 7 may be in range of 0.4-0.8 inches. However, of course, dimensions of the extension 7 may be varied as desired.

Further, according to aspects of the disclosure, the extension 7 could be configured, in combination with the length of the tip 5, as a guide to aid the user in applying to the substance to an intended area. For example, in the above described embodiment, wherein the applicator 1 is configured as a nasal applicator, the extension 7 may be positioned a predetermined distance from the end of the tip 5, such that the portion of the tip 5 which is coated with the substance will be positioned in the appropriate place (e.g., 0.25 inches inside the nostril) when the extension is in contact with the outer portion of the nose.

It is noted that, as seen in FIG. 1A, the extension 7 may be joined with the breakaway section 9. Aspects of the attachment between the breakaway section 9 and the extension 7 will be described in further detail below. However, it is noted here that the extension 7 may be configured with sufficient rigidity to withstand the force applied by the user to sever the breakaway section 9 from the extension 7. According to aspects of the disclosure, the extension 7 may be made of a material and of a size sufficient to withstand the force applied by the user to sever the breakaway section 9 from the extension 7. For example, according to aspects of the disclosure, the thickness of the extension 7 (e.g., the size of the extension in the right to left direction shown in FIG. 1A) may be in a range of 0.1-0.2 inches. However, of course, the dimensions of the extension 7 may be varied as desired.

According to aspects of the disclosure, and as seen in FIG. 1A, the tip 5 may be an elongated member (e.g., a cylindrical member) which is joined with, or integral with, the handle 3 and the extension 7. For example, as seen in FIG. 1A, according to aspects of the disclosure, the tip 5 may extend from the handle 3 in an area proximate to where the extension 7 extends from the main body 2. As will be described in detail below, the tip 5 may function as an applicator stick through which the substance (which may be coated onto the tip 5) is applied to an intended surface. The elongated and cylindrical configuration described above may aid the user is in maneuvering the tip 5 in applying the substance. For example, the tip 5 may be rotated radially in order to transfer the substance from the circumference of the tip 5 to the intended surface to which the substance is to be applied.

As mentioned above, according to aspects of the disclosure, the tip 5 of the applicator 1 may hold a particular amount of a substance to be applied to an intended surface. According to some aspects of the disclosure, the tip 5 may be coated with the substance. The amount of substance with which the tip 5 is coated may vary as desired. For example, according to aspects of the disclosure, the tip 5 is partially coated with the substance (e.g., only the upper half of the length of the tip 5 is coated with the substance). According to other aspects of the disclosure, the tip 5 is substantially or entirely coated with the substance.

It is noted that according to aspects of the disclosure, the applicator 1 may be filled by inverting the applicator 1 (e.g., with the handle 3 facing downward and the breakaway section 9 and the tip 5 both facing upward). Thereafter, the substance is applied to the tip 5 of the applicator 1. According to aspects of this disclosure, a controlled dose of the substance may be placed onto the tip 5 of the applicator 1. Once the controlled dose is on the tip 5 the applicator 1 may be sealed. For example, the end of the breakaway section 9 may be heat sealed via a crimping means. FIG. 1B shows a perspective view of the applicator 1 once the applicator 1 has been filled and sealed and the end of the breakaway section 9a has been heat crimped. It is noted, that the above described process for forming the applicator 1 is merely an example of one such process by which the applicator may be formed and, of course, different variations of the process or other processes may be used.

According to some aspects of the disclosure, the amount of substance 10 on the tip is approximately equal to the desired amount of substance 10 to be dispensed (i.e., the accurate and controlled dose). Although, it is noted that according to other aspects of the disclosure, the amount of substance 10 on the tip 5 may be slightly more than the accurate and controlled dose (e.g., 1-10% more). According to aspects of the disclosure, the amount of substance 10 on the tip 5 may be in the range of 0.05 fluid milliliters.

According to aspects of the disclosure, the tip 5 may include rounded edges. For example, in the embodiment shown in FIG. 1A, the tip 5 tapers inwardly as it extends away from the handle 3 so as to form a curved, gently rounded, or conical shape at the end of the tip 5 furthest from the handle 3. It is noted that according to aspects of the disclosure wherein the applicator 1 is an applicator designed for applying substance into the nasal cavity, such a cylindrical configuration with a curved, gently rounded, or conical shape at the end of the tip 5 may be advantageous, because it will provide comfort and prevent, or minimize the risk of, discomfort or injury during use. However, the above described example is merely an illustrative embodiment and according to other embodiments of the disclosure (e.g., such as those embodiments described below), the tip 5 may be configured differently.

According to aspects of the disclosure, the applicator 1 may have a tip 5 with a diameter, or thickness, in the range of 0.2 inches and 0.5 inches. According to aspects of the disclosure, the length of the tip 5 may be in a range of 0.25-0.75 inches. However, it is noted that, of course, the dimensions may be varied as desired. For example, the length of tip 5 (e.g., the length of the tip 5 as measured from the where it meets the extension 7) may be varied depending on the particular environment in which it is intended to be used. For example, according to aspects of the disclosure, the applicator may be intended to apply a substance into the nasal cavity. In such an embodiment the length of tip 5 (e.g., the length of the tip 5 as measured from the where it meets the extension 7) should be chosen such that it will prevent over penetration of the tip 5 into the nasal cavity. For example, the length of the tip 5 may be in a range of 0.4-0.5 inches. Further, according to aspects of the disclosure, the tip 5 may be configured with a length that allows an adequate amount of the substance to be applied to the tip 5.

Further, according to aspects of the disclosure, the tip 5 may be configured with a length such that once the substance is applied to the tip 5, depending on the viscosity of the substance, the substance may migrate slightly along the tip 5 in a direction towards the handle 3 without reaching the end of the tip 5 near the extension.

As discussed above, the tip 5 may be made of a plastic material such as polypropylene, high density polyethylene, low density polyethylene, polyethylene terephthalate (PET) or some other type of plastic. By having the tip 5 comprised of a plastic material, it will prevent the substance from being absorbed into the tip 5. For example, once the tip 5 is coated with the substance, the substance will merely adhere to the outer surface of the tip 5. In contrast, conventional applicators which have swabs that are comprised of cotton, fabric, etc., may allow the substance to be absorbed into the cotton, fabric, etc. This may be especially true if the conventional applicator has a relatively long storage period. If the substance is absorbed into the swab of the conventional applicator, then the substance is less likely to be applied to the intended surface during use (e.g., it may be more difficult to ensure that the proper dosage of the substance is applied to the intended surface during use). For example, as discussed above, in some cases, a large amount of the controlled dose that is desired to be dispensed may remain in the swab of the applicator.

Hence, it is understood that making the tip 5 from a plastic material will increase the likelihood that the proper dosage of the substance is applied to the intended surface because the substance will not be absorbed into the tip 5. Further, having a plastic tip 5 will increase the likelihood that the proper dosage of the substance is applied to the intended surface because the substance will be less likely to adhere or stick to the tip 5 during use than a swab of cotton, fabric, etc.

Of course, according to other aspects of the disclosure, the tip 5 may be made from other materials as well (e.g., metals, rubber, composites, alloys, etc.) which would provide the above described benefits as well. In other words, such materials would prevent penetration (e.g., absorption) of the substance into the tip 5 and, therefore, increases likelihood of correct dosage of substance being applied during use. However, the above discussion is not intended to imply that a non-absorbent material must be used to create the tip 5. Instead, according to aspects of the disclosure, materials other than plastic, metal, rubber, composites, alloys, etc. may also be used to create the tip 5.

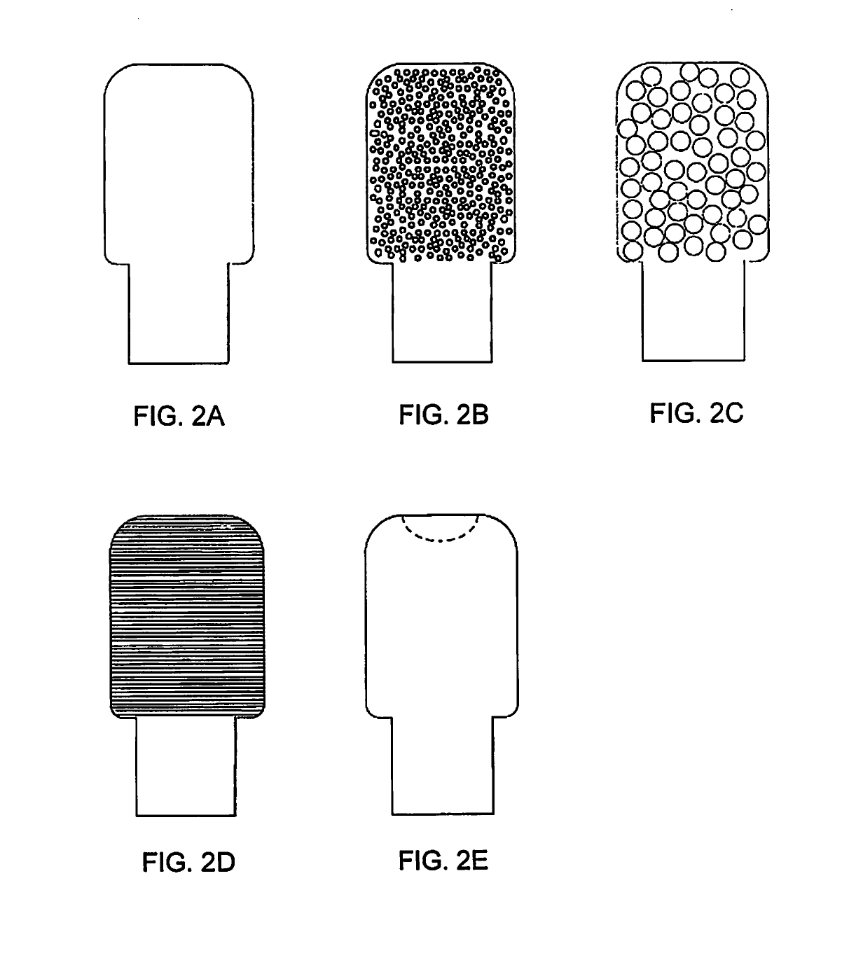

According to aspects of the disclosure, the tip 5 may be configured to aid the substance adhere to the tip 5, and/or aid the substance to be transferred from the tip 5 to the intended surface during use. For example, according to aspects of the disclosure, the tip 5 may include a smooth surface. For example, according to aspects of the disclosure, the tip 5 may be devoid of any cracks, fissures, etc. FIG. 2A shows an embodiment of the tip 5 wherein the tip 5 includes a smooth surface.

According to aspects of the disclosure, the tip 5 may include a roughened surface. For example, according to aspects of the disclosure, the tip 5 may have been exposed to a surface treatment in order to create a roughened surface on the tip 5. For example, according to aspects of the disclosure, the surface of the tip 5 may have been exposed to blasting. For example, according to aspects of the disclosure, the tip 5 may have been blasted with relatively small particles such as sand, metal beads, glass beads, rough glass, etc. Exposing the tip 5 to such surface treatments, may configure the tip 5 to aid the substance adhere to the tip 5, and/or aid the substance to be transferred from the tip 5 to the intended surface during use. FIG. 2B is an illustrative embodiment wherein the tip 5 has been exposed to sandblasting. As seen in FIG. 2B, the tip 5 includes a plurality of relatively small recesses. FIG. 2C is an illustrative embodiment wherein the tip 5 has been exposed to blasting by metal beads.

According to aspects of the disclosure, the surface of the tip 5 may have been exposed to etching in order to create a roughened surface. For example, according to aspects of the disclosure, the tip 5 may have been exposed to chemical etching, laser etching, etc. Exposing the tip 5 to such surface treatments, may configure the tip 5 to aid the substance adhere to the tip 5, and/or aid the substance to be transferred from the tip 5 to the intended surface during use. FIG. 2D is an illustrative embodiment wherein the tip 5 has been exposed to etching. As seen in FIG. 2D, the tip 5 includes a plurality of recesses or grooves. It is noted, that while the illustrative embodiment shown in FIG. 2D includes relative straight recesses or grooves that extend around the circumference of the tip, according to other aspects of the disclosure, the etching could provide any desired configuration. For example, according to aspects of the disclosure, the etching could provide recesses or grooves that are diagonally oriented, non-uniform, oriented in different direction relative to each other, etc.

According to aspects of the disclosure, the tip 5 may include a recess or concave portion on the terminal end of the tip 5. The recess or concave portion may serve as a reservoir for at least a portion of the substance. Such a configuration of the tip 5 may aid the substance in adhering to the tip 5, and/or aid the substance in being transferred from the tip 5 to the intended surface during use. FIG. 2E is an illustrative embodiment of such a tip 5 with a recess or concave portion on the top of the tip 5. It is noted that a recess or concave portion may be positioned on other parts of the tip 5 as well.

As described above, according to aspects of the disclosure, the tip 5 may be made of a plastic such as rubber. For example, according to aspects of the disclosure the tip 5 may be made of NEOPRENE® (manufactured by E. I. du Pont de Nemours and Company of Wilmington, Del.) or other similar synthetic rubber materials. As described above, according to aspects of the disclosure, the applicator 1 may be manufactured of more than one material. Hence, according to aspects of the disclosure, the applicator 1 may include a handle 3, extension 7 and breakaway section 9 made of polypropylene, high density polyethylene, low density polyethylene, polyethylene terephthalate (PET), etc. and a tip 5 (or a portion of the tip 5) comprised of NEOPRENE® or other rubber materials. For example, according to aspects of the disclosure, the applicator 1 may be created via a co-injection molding process wherein the different materials are co-injected (e.g., double shot).

According to aspects of the disclosure the tip 5 may be made of polypropylene, high density polyethylene, low density polyethylene, polyethylene terephthalate (PET), etc., and coated in NEOPRENE or another rubber material. For example, according to aspects of the disclosure, the NEOPRENE® or other rubber material may extend over a portion of the tip 5 (e.g., a portion at the upper half of the tip 5) or alternatively, extend over the entire or substantially the entire tip 5.

As described above, according to aspects of the disclosure, the applicator 1 may include a breakaway section 9. The breakaway section 9 is primarily useful in preventing contamination of the substance 10 which coats the tip 5. Additionally, the breakaway section 9 may also be useful containing the substance 10 in the applicator 1 until the substance 10 is to be dispensed or applied. According to aspects of the disclosure, and as seen in FIG. 1A, the breakaway section 9 may be an elongated tube or sleeve (e.g., a cylindrical tube or sleeve). Further, according to aspects of the disclosure, the breakaway section 9 may be removably attached to the remainder of the applicator 1 at the extension 7. For example, as seen in FIG. 1A, according to aspects of the disclosure, the breakaway section 9 may be removably attached to the extension 7 in an area proximate to a terminal end of the extension 7 (i.e., the end of the extension 7 that extends away from the main body 2).

According to aspects of the disclosure, and as seen in FIG. 1A, the breakaway section 9 may have a diameter larger than the diameter of the tip 5 and, further, large enough to provide a clearance both around the circumference of the tip 5 and the end of the tip 5 that is farthest from the handle 3. For example, according to aspects of the disclosure, the breakaway section 9 may have a diameter of 0.4-0.8 inches. Further, according to aspects of the disclosure, the length of the breakaway section 9 may be in a range of 0.5-1.5 inches. However, of course, the dimensions of the breakaway section 9 may be varied as desired.

According to aspects of the disclosure, the breakaway section 9 may be relatively flexible or relatively rigid. For example, according to aspects of the disclosure, it would be desirable for the structural integrity of the breakaway section 9 provides sufficient rigidity to prevent the breakaway section 9 from being compressed in a way in which the inner edge of the breakaway section 9 contacts the substance on the tip 5 such that the substance on the tip 5 is transferred to the breakaway section 9.

As described above, according to some embodiments of this disclosure, the wall thickness of the breakaway section 9 may be varied in order to provide appropriate rigidity. For example, as described above, according to some aspects of this disclosure, breakaway section 9, or portions thereof, may be made of a plastic material such as polypropylene, high density polyethylene, low density polyethylene, polyethylene terephthalate (PET) or some other type of plastic and the wall thickness may be made an appropriate dimension in order to provide the desired rigidity. Of course, according to other aspects of the disclosure, the breakaway section 9 may be made from other materials as well.

Further, according to some embodiments of the disclosure, and as seen in FIG. 1A, in order to make the breakaway section 9 easier to detach from the applicator 1, the wall thickness of the portion of the breakaway section 9 that is near the extension 7 may be less thick than the wall thickness of the remainder of the breakaway section 9. Hence, the thinner portion of the breakaway section 9, may be less rigid than the remainder of the breakaway section 9 and, thereby, more susceptible to being fractured. As a result, when the user applies a force (e.g., a torsion force) to the breakaway section 9 in order to detach the breakaway section 9 from the applicator 1, the thinner portion of the breakaway section 9 is readily breakable.

According to aspects of the disclosure, the breakaway section 9 may have a first predetermined wall thickness. Further, according to aspects of the disclosure, the breakaway section 9 may have a section with a thinner wall thickness near the extension 7. However, of course, the dimensions of the wall thickness of the breakaway section 9 (including the thicker and thinner sections) may be varied as desired.

According to aspects of the disclosure, this thinner section of the breakaway section 9 may comprise, at least in part, a separation section 11 of the applicator 1. The separation section 11 of the applicator 1 may refer to the area of the applicator 1 wherein the breakaway section 9 is detached from the applicator 1. For example, in the embodiment shown in FIG. 1A, the separation section 11 of the applicator 1 is positioned at the area where the breakaway section 9 meets the extension 7.

According to the aspects of the disclosure, the separation section 11 may be frangible such that the breakaway section 9 is easily detachable from the applicator 1. As described above, the separation section 11 may be formed as merely a thinner wall section of the breakaway section 9. For example, as seen in FIGS. 1A and 1B, the separation section 11 may comprise a recess or groove which extends around the circumference of the applicator 1. For example, the recess or groove may extend 360 degrees around the breakaway section 9 where the breakaway section 9 meets the extension 7. However, according to other embodiments, the separation section 11 may be formed in other ways. For example, according to aspects of the disclosure, the breakaway section 9 may have a uniform wall thickness and the separation section 11 may be formed as a series of perforations into the breakaway section 9. For example, the series of perforations may extend 360 degrees around the breakaway section 9 where the breakaway section 9 meets the extension 7. Additionally, the separation section 11 may be configured in other ways as well in order to ensure that the separation section 11 is frangible and the breakaway section 9 is easily detachable from the applicator 1.

According to aspects of the disclosure, the breakaway section 9 may be sealed at its end that is farthest from the handle 3. For example, according to aspects of the disclosure, after the applicator 1 has been filled with the substance (e.g., after the tip has been coated with the substance), the breakaway section 9 may be heat crimped at its end that is farthest from the handle 3 in order to seal the breakaway section 9 and, thereby, the applicator 1. FIG. 1B illustrates a perspective view of an applicator 1 according to aspects of the disclosure wherein the breakaway section 9 has been sealed in such a fashion. In this way, the breakaway section 9 contains the substance 10 in the applicator 1 until the substance 10 is to be dispensed or applied and, further, prevents contamination of the substance 10 which coats the tip 5. It is noted that the breakaway section may be sealed according to other methods as well. Further, according to other embodiments, the breakaway section 9 may be configured as a cap (e.g., resembling a cap for a pen wherein the breakaway section 9 is relatively cylindrical and, also, closed at its end furthest from the handle 3).

According to aspects of the disclosure, the breakaway section 9 and the extension may be configured so that after the breakaway section 9 is detached from the surface of the extension 7, the surface of the extension 7 is relatively smooth. In other words, according to some aspects of the disclosure, the separation section 11 is configured such that the extension 7 does not have any jagged or sharp edges after the breakaway section 9 has been detached from the applicator 1.

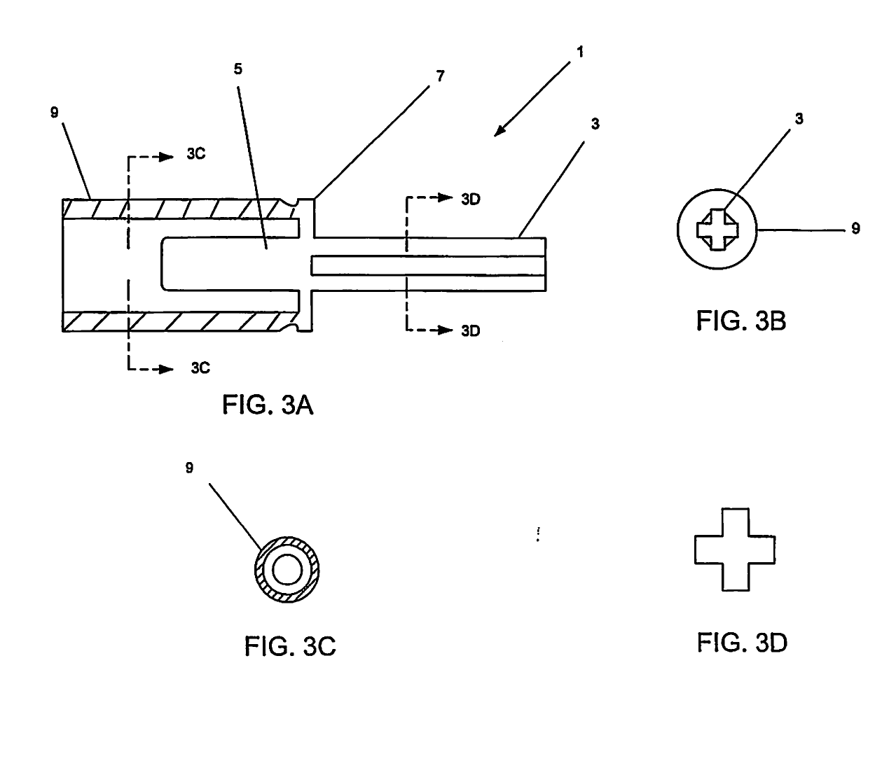

FIGS. 3A-3D illustrate various views of another embodiment of an applicator according to at least one aspect of the disclosure. FIG. 3A illustrates an applicator 1 according to at least one aspect of the disclosure wherein portions of the applicator 1 are shown in cross-section for illustrative purposes. FIG. 3B illustrates an end view of the applicator 1 shown in FIG. 3A when viewed from the handle 3 towards the tip 5. FIG. 3C illustrates a sectional view of the applicator 1 shown in FIG. 3A along the line 3C-3C in FIG. 3A. FIG. 3D illustrates a sectional view of the applicator 1 shown in FIG. 3A along the line 3D-3D in FIG. 3A.

Many of the features in the embodiment shown in FIGS. 3A-3D are similar to features described above and, therefore, for the sake of brevity will not be elaborated on here in detail. However, it is noted that in this embodiment of the applicator 1, as seen in FIG. 3A, the tip 5 has a more rectangular shape but exhibits curved or rounded edges at the portion of the tip 5 farther from the handle 3. Further, it is noted that according to aspects of the disclosure, and as seen most clearly in FIG. 3D, the handle 3 may be shaped as a cross with four arms extending outwards. Such a configuration may aid the user in gripping the handle 3.

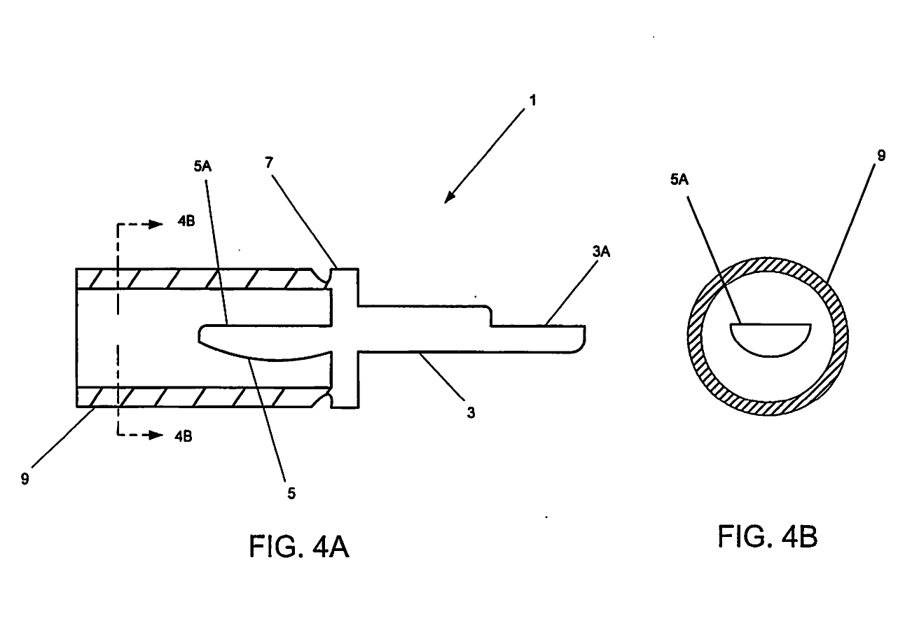

FIGS. 4A and 4B illustrate another embodiment of an applicator 1 according to at least one aspect of the disclosure. FIG. 4A illustrates an applicator 1 according to at least one aspect of the disclosure wherein portions of the applicator 1 are shown in cross-section for illustrative purposes. FIG. 4B illustrates a sectional view of the applicator 1 shown in FIG. 4A along the line 4B-4B in FIG. 4A. Many of the features in the embodiment shown in FIGS. 4A and 4B are similar to features described above and, therefore, for the sake of brevity will not be elaborated on here in detail.

However, it is noted that in this embodiment of the applicator 1, as seen in FIGS. 4A and 4B, the tip 5 includes a flat surface, or edge 5A. According to aspects of the disclosure, the flat edge 5A may extend across the width of the tip 5, such that in the orientation shown in FIGS. 4A and 4B, the entirety (or substantially the entirety) of the upper section of the tip 5 would be flat. According to aspects of the disclosure, the flat edge 5A may be the surface of the tip 5 which holds the substance. In other words, according to aspects of the disclosure, the flat edge 5A may be the portion of the tip 5 which is coated with the substance that is to be applied. Hence, in such embodiments, the flat edge 5A may be the portion of the tip 5 that is to be used to apply the substance to an intended surface.

In such embodiments wherein the flat edge 5A is the portion of the tip 5 that is to be used to apply the substance to an intended surface, during use, if desired, the flat edge 5A of the applicator 1 may be manipulated as a scraper or spreader to transfer the substance to the intended surface. For example, in the embodiment of the disclosure wherein the applicator 1 is configured for applying the substance into the nasal cavity, the flat edge 5A of the applicator 1 may be manipulated to scrape and/or spread the substance to the interior of the nasal cavity. For example, the flat edge 5A of the applicator 1 may be manipulated to scrape and/or spread the substance on the interior of the outer portion of the nostril.

It is noted that, as discussed with reference to the embodiments above, the tip 5 of the applicator 1 may include rubber (e.g., NEOPRENE® or another rubber material). For example, flat edge 5A may be coated with NEOPRENE® or another synthetic rubber material. Therefore, the flat edge 5A may function as a squeegee to apply the substance to the intended surface. It is also noted that other features of the embodiments described above (e.g., surface treatments) may be applied to this embodiment as well.

According to aspects of the disclosure, and as seen in FIG. 4A, the tip 5 may exhibit curved or rounded edges at the portion of the tip 5 that is farthest from the handle 3. Additionally, as seen in FIGS. 4A and 4B, the lower portion of the tip 5 (relative to the orientation shown in FIGS. 4A and 4B) may be substantially cylindrical. Also, as seen in FIGS. 4A and 4B, according to aspects of the disclosure, the lower portion of the tip 5 may be configured such that the middle of the lower portion is bowed relative to the ends of the lower portion (e.g., the middle of the lower portion has a larger thickness than the ends of the lower portion).

It is noted that the lower portion of the tip 5 may be configured as desired. However, according to aspects of the disclosure, it may be configured differently than the flat edge 5A of the upper portion of the tip 5. By configuring the lower portion of the tip 5 differently from the flat edge 5A of the tip 5, the tip 5 itself may function as an indicator to indicate which edge of the tip 5 holds the substance and further, which edge of the tip is to be used to apply the substance to the intended surface.

As noted, the above described embodiment of the applicator 1 may be particularly advantageous for transferring a substance into the nasal cavity. For example, in circumstances wherein a controlled dose of a substance is to be applied to a nasal cavity, the substance may be absorbed too rapidly if it is applied to the interior of the nasal cavity (the portion of the interior of the nasal cavity that is opposite the interior of the nostril walls). The membranes on such portions of the nasal cavity may absorb the substance much more quickly than the other portions of the nasal passageway (e.g., the interior of the outer wall of the nostril).

Accordingly, the embodiment of the applicator 1 described above may be configured to prevent the substance from being absorbed too quickly by the nasal cavity. For example, the embodiment of the applicator 1 described above includes a tip 5 with a flat edge 5A onto which the substance is coated and a rounded, lower edge opposite the flat edge 5A. Such a configuration of the application is designed to allow the applicator 1 to function properly when the flat edge 5A of the applicator 1 is manipulated to scrape and/or spread the substance on the interior of the outer portion of the nostril while the rounded, lower edge opposite the flat edge 5A would correspond to the interior of the nasal cavity opposite the nostril wall. Therefore, it is understood that such an applicator 1, according to aspects of the disclosure, may be configured to apply a substance to a portion of the interior of the outer wall of the nostril and, hence, prevent the substance from be absorbed too rapidly.

According to aspects of the disclosure, the applicator 1, as seen in FIGS. 4A and 4B, may include a handle 3 with a flat edge 3A. According to aspects of the disclosure, the flat edge 3A may extend across the width of the handle 3, such that in the orientation shown in FIGS. 4A and 4B, the entirety (or substantially the entirety) of the upper section of the handle would be flat. According to aspects of the disclosure, the flat edge 3A may be the surface of the handle 3 and may correspond to the flat edge 5A of the tip 5. For example, as seen FIG. 4A, the flat edge 3A of the handle 3 may be oriented such that it faces the identical direction as the flat edge 5A of the tip 5. In this way, the flat edge 3A of the handle 3 may indicate to the user the orientation of the flat edge 5A of the tip 5. Hence, the user would be able to grip the handle 3 in such a way that it would be easy to manipulate the flat edge 5A of the tip 5 to apply the substance to the intended surface. For example, in embodiments of the disclosure wherein the applicator 1 is configured for applying the substance into the nasal cavity and the flat edge 5A is the portion of the tip 5 that is to be used to apply the substance to an intended surface (e.g., the interior of the outer portion of the nostril), the flat edge 3A would indicate to the user how to grip the handle such that during use, such that, if desired, the flat edge 5A of the applicator 1 may be manipulated as a scraper or spreader to transfer the substance to the intended surface.

According to aspects of this disclose, the applicator 1 may include one or more indicators which indicate information to the user. For example, the indicators may be visual, sensory (e.g., felt through touch), etc. As described above, the configuration of the tip 5 (e.g., the flat edge 5A of the tip 5 in contrast to the rounded, lower portion of the tip 5) and/or the configuration of the handle 3 (e.g., the flat edge 3A of the handle 3 in contrast to the rounded, lower portion of the handle 3) may be a visual indicator to the user as to which edge of the tip 5 contains the substance and is to be applied to the intended surface in order to transfer the substance to the intended surface.

According to aspects of the disclosure, the handle 3 may include additional indicators as well. For example, according to aspects of the disclosure, the handle 3 may include engraving. For example, the flat edge 3A of the handle 3 may include engraving. For example according to one embodiment, the engraving may include words or phrases that convey information to the user, such as “grip here” or the expiration date of the substance in the applicator 1. Of course, other indicators may be used as well. For example, alternatively, or, in addition to, engraving, the handle 3 of the applicator 1 may include raised markings. For example, the phrases described above may be configured with raised lettering or Braille. Of course, it is understood that any designs, phrases, etc. may be used as desired.

According to aspects of the disclosure, color may be used as an indicator as well. For example, according to aspects of the disclosure, a portion of the handle 3 may be created with a particular color (e.g., orange) while the rest of the handle is a color different from the particular color (e.g., white). In such an embodiment, the user may quickly determine which side of the applicator 1 to grip and, further, which side of the applicator is to be applied to the intended surface to transfer the substance to the intended surface. It is noted that color may be particularly useful as an indicator when the handle 3 and/or tip 5 is not configured so as to be easily identifiable as to which edge of the tip 5 contains the substance and, therefore, is to be applied to the intended surface to transfer the substance to the intended surface. For example, color may be particularly useful on one side of the handle if the handle is symmetrical (e.g., shaped like a tongue depressor or popsicle stick). However, color may be used with other configuration as well.

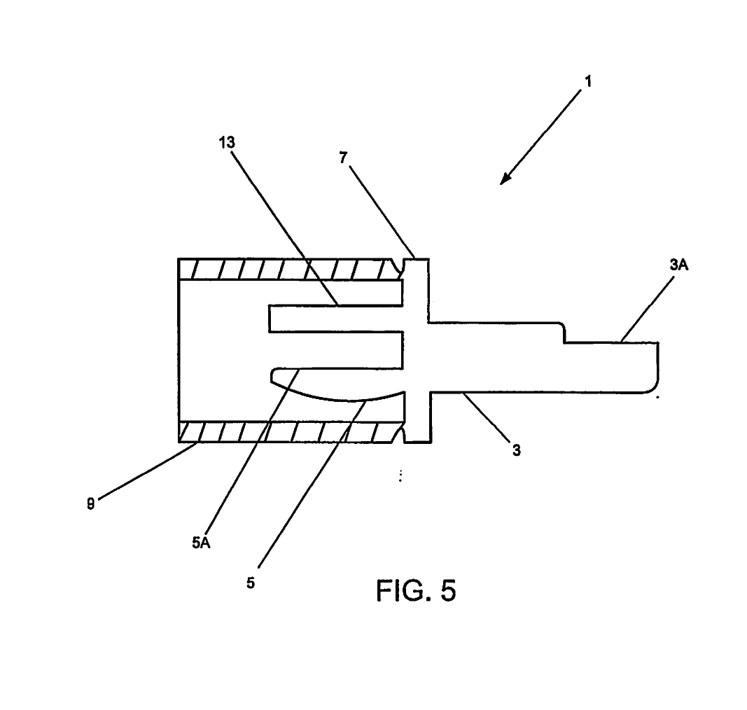

FIG. 5 illustrates another embodiment of an applicator 1 according to at least one aspect of the disclosure. FIG. 5 illustrates an applicator 1 according to at least one aspect of the disclosure wherein portions of the applicator 1 are shown in cross-section for illustrative purposes. Many of the features in the embodiment shown in FIG. 5 are similar to features described above and, therefore, for the sake of brevity will not be elaborated on here in detail. However, it is noted that in this embodiment of the applicator 1, as seen in FIG. 5, the applicator 1 includes a member 13 which extends from the main body 2.

According to aspects of the disclosure, and as seen in FIG. 5, the member 13 may be an elongated member (e.g., a cylindrical member) which is joined with, or integral with, the handle 3 and the extension 7. For example, as seen in FIG. 5, according to aspects of the disclosure, the member 13 may extend from the handle 3 in an area proximate to where the extension 7 extends from the main body 2.

According to aspects of the disclosure, the member 13 may have a diameter, or thickness, in the range of 0.2 inches and 0.5 inches. According to aspects of the disclosure, the length of the member 13 may be in a range of 0.25-0.75 inches. However, it is noted that, of course, the dimensions may be varied as desired. For example, the length of member 13 (e.g., the length of the member 13 as measured from where it meets the extension 7) may be varied depending on the particular environment in which it is intended to be used. For example, according to aspects of the disclosure, in the embodiments wherein the applicator 1 is configured to apply a substance into the nasal cavity the length of the tip 5 may be in a range of 0.4-0.5 inches.

As will be described in detail below, the member 13 may function as a guide that the user may use in applying the substance (which may be coated onto the tip 5) to an intended surface. For example, according to aspects of the disclosure, the member 13 may be positioned opposite the tip 5. For example, as seen in FIG. 5, the member 13 may be positioned opposite the tip 5 in order to form a U-shape or horseshoe shape. Further, as seen in FIG. 5, the member 13 may include a flat edge. According to other embodiments the member may include a slightly concave edge. Further, as seen in FIG. 5, the member 13 may be oriented such that it is opposite the flat edge 5A of the tip 5 (e.g., the flat or slightly concave edge of the member may face the flat edge 5A of the tip 5). It is noted that the U or horseshoe shape may be configured for particular purposes. For example, the member 13 which forms a portion of the U or horseshoe shape may act as a reference guide. For example, the U or horseshoe shape may be configured such that the tip 5 and the member 13 surround an opposite edge of a wall which contains the intended surface onto which the substance is to be applied. Further, the member 13 may be configured to align against the outside edge of a wall while the opposite side of the wall (i.e., the side of the wall facing away from and not in contact with member 13) may be the intended surface to which the substance is to be applied. Therefore, the user may manipulate the applicator 1 such that the horseshoe or U shape is arranged as described above and, hence, the member 13 may contact the wall and/or otherwise act as a reference guide while the tip 5 applies the substance to the intended surface.

According to aspects of the disclosure, the gap between the member 13 and the tip 5 may be sized for various purposes. For example, in the embodiments wherein the applicator 1 is configured as an applicator 1 for applying a substance within the nasal cavity, the gap between the member 13 and the tip 5 may be sized such that the wall of the nostril would fit between the member 13 and the tip 5. For example, according to aspects of the disclosure, the gap between the member 13 and the tip 5 may be in a range of 0.25 inches to 0.5 inches.

It is noted, that as seen in FIG. 5, the member 13 and the tip 5 may be configured differently to differentiate between the tip 5 and the member 13. For example, as seen in FIG. 5, the member 13 and the tip 5 are shaped differently with the member being more rectangular and the tip 5 exhibiting a more curved shape. Of course, alternatively, or in addition to, different configurations of the member 13 and the tip 5, other indicators such as described above (e.g., engraving, colors, etc.) may be used to differentiate the member 13 and the tip 5.

According to aspects of the disclosure, while not shown it is noted that portions of the applicator may be configured with recesses or other configurations that are sized to receive a typical finger and/or thumb. For example, portions of the handle 3 or breakaway section 9 may be configured with recesses or other configurations that are sized to receive a typical finger and/or thumb. Additionally, it is noted that the recesses may be oriented for the particular use. For example, some of the recesses may be configured to provide the user with a point to grip the applicator 1 for removing the breakaway section from the applicator 1, while alternatively, or in addition to, some of the recesses may be configured to provide the user with a point to grip the applicator 1 during the application of the substance to the intended surface. Of course, other configurations that may increase traction between the portions of the applicator 1 and the user's fingers (e.g., ridges) or may aid the user in realizing a “proper” positioning of their fingers (e.g., a positioning that makes it relatively easy to manipulate the applicator 1 for an intended purpose) may be used as well.

It is noted that in the embodiments described above, the tip 5 of the applicator 1 is shown as extending in a direction parallel to the handle 3. However, this is not meant to imply that the tip 5 must extend in such a direction. Instead, according to aspects of the disclosure, the tip 5 may be positioned at an angle in relation to the handle 3. For example, the tip 5 may be oriented at an angle of 15°, 30°, 40°, etc. relative to the handle 3.

As mentioned above, particular aspects of this disclosure may relate to a dispensing apparatus configured to be used as an applicator 1 for applying a substance into the nasal cavity. Particular substances that may be used in conjunction with such an embodiment of the disclosure may include: NASOBOL (lira-nasal Testoserone), ANDRODERM, NOSEAFIX, Bepotastine, Civamide, Ereska, FluNsure, Intranasal Diazepam, Midazoam, Morphine Gluconate, Nasal LORAZEPAM, NASCOBAL, Pieconaril, Rylomine, and SinuNase.

While the disclosure has been described with respect to specific examples including presently preferred modes of carrying out the disclosure, those skilled in the art will appreciate that there are numerous variations and permutations of the above described structures and methods. For example, of course, the above recited dimensions are not intended to be limiting and the dimensions of a particular applicator device according to aspects of this disclosure could be varied based on the particular purpose for which that particular applicator is intended. Further, it is noted that the embodiments described herein are merely illustrative. Of course, numerous other embodiments are contemplated and considered within the scope of the disclosure. For example, the particular measurements recited in this disclosure and in the drawings describe a single embodiment of the disclosure. Many other embodiments with different dimensions (e.g., different lengths, different volume capacities, etc.) are considered within the scope of the disclosure. Thus, the spirit and scope of the disclosure should be construed broadly.

1. An applicator for applying a substance to an intended surface comprising:
a main body including:
an elongated handle; and
an elongated tip configured to hold a predetermined amount of the substance;
an extension which extends radially from the main body in an area proximate to where the handle meets the tip; and
a breakaway section including an elongated sleeve which extends in a direction perpendicular to at least a portion of the tip and extends from the extension in an area proximate to a terminal end of the extension,
wherein the elongated tip extends from the handle in an area proximate to where the extension extends from the main body,
wherein the applicator is a single, integral piece such that the handle, the elongated tip, the extension and the breakaway section are integral with each other.

2. The applicator of claim 1, wherein the single, integral piece applicator is made from a material which does not absorb the substance.

3. The applicator of claim 2, wherein the material from which the applicator is created is a plastic.

4. The applicator of claim 3, wherein the material from which the applicator is created is one of: polypropylene, high density polyethylene, low density polyethylene and polyethylene terephthalate (PET).

5. The applicator of claim 1, wherein at least a portion of the tip has been subjected to a surface treatment.

6. The applicator of claim 5, wherein the surface treatment is one of: sandblasting, blasting with glass or metal beads, blasting with rough glass, chemical etching and laser etching.

7. The applicator of claim 1, wherein the elongated tip has a cylindrical configuration which tapers inwardly as the tip extends from the handle and forms a curved, gently rounded, or conical shape at the end of the tip furthest from the handle.

8. The applicator of claim 1, wherein the extension includes a disk extending substantially around the circumference of the main body.

9. The applicator of claim 8, wherein the extension is sized to prevent the handle from entering a human nasal cavity.

10. The applicator of claim 1, wherein the tip includes a concave or recess at the terminal end of the tip.

11. The applicator of claim 1, further comprising a recess or groove which extends around the circumference of the applicator in an area proximate to where the breakaway section meets the extension, wherein the recess or groove creates a frangible section of the breakaway section wherein a wall thickness of the frangible section of the breakaway section is thinner than a wall thickness of the majority of the breakaway section.

12. The applicator of claim 1, wherein at least a portion of the elongated tip is coated in a rubber material.

13. The applicator of claim 1, wherein at least a portion of the elongated tip is angled with respect to the handle.

14. An applicator for applying a substance to an intended surface comprising:
a main body including:
an elongated handle with a flat surface; and
an elongated tip with a flat surface configured to hold a predetermined amount of the substance;
an extension which extends radially from the main body in an area proximate to where the handle meets the tip; and
a breakaway section including an elongated sleeve which extends in a direction perpendicular to at least a portion of the tip and extends from the extension in an area proximate to a terminal end of the extension,
wherein the elongated tip extends from the handle in an area proximate to where the extension extends from the main body,
wherein the flat surface of the handle and the flat surface of the elongated tip face the same direction and correspond to each other such that the flat surface on the handle indicates the direction that the flat surface of the elongated tip faces.

15. The applicator of claim 14, wherein the flat surface extends across a width of the elongated tip.

16. The applicator of claim 14, wherein at least a portion of the flat surface is coated with a rubber material.

17. An applicator for applying a substance to an intended surface comprising:
a main body including:
an elongated handle;
an elongated tip configured to hold a predetermined amount of the substance; and
an elongated member;
an extension which extends radially from the main body in an area proximate to where the handle meets the tip; and
a breakaway section including an elongated sleeve which extends in a direction perpendicular to at least a portion of the tip and extends from the extension in an area proximate to a terminal end of the extension,
wherein the elongated tip and the elongated member both extend from the handle in an area proximate to where the extension extends from the main body to form a U or horseshoe shape with a gap extending between the elongated tip and the elongated member.

18. The applicator of claim 17, wherein the elongated member is configured differently from the elongated tip.

19. The applicator of claim 17, wherein the elongated tip includes a flat surface configured to hold a predetermined amount of the substance and the elongated member includes a flat or concave surface that faces the flat surface of the elongated tip.

20. The applicator of claim 17, wherein at least a portion of the flat surface is coated with a rubber material.
